(19) United States
(12) Patent Application Publication
(10) Pub. No.: US 2006/0161219 A1
(43) Pub. Date: Jul. 20, 2006

(54) Electrical stimulation system and method for stimulating multiple locations of target nerve tissue in the brain to treat multiple conditions in the body

(75) Inventors: W. Alan Mock, Frisco, TX (US); John H. Erickson, Plano, TX (US); Tracy L. Cameron, Toronto (CA); Christopher G. Chavez, McKinney, TX (US)

Correspondence Address: BAKER BOTTS L.L.P., 2001 ROSS AVENUE, 6TH FLOOR, DALLAS, TX, 75201-2980, US

(73) Assignee: Advanced Neuromodulation Systems, Inc.

(21) Appl. No.: 11/335,436

(22) Filed: Jan. 18, 2006

Related U.S. Application Data
(63) Continuation-in-part of application No. 10/994,008, filed on Nov. 18, 2004.
(63) Continuation-in-part of application No. 11/207,086, filed on Aug. 18, 2005.
(60) Provisional application No. 60/645,405, filed on Jan. 19, 2005.
(60) Provisional application No. 60/523,710, filed on Nov. 20, 2003.
(60) Provisional application No. 60/602,522, filed on Aug. 18, 2004.

(51) Int. Cl. A61N 1/34

(52) U.S. Cl. 607/45

(57) ABSTRACT

An electrical stimulation system is provided for electrically stimulating nerve tissue in multiple locations in a person's brain to treat multiple conditions in the person's body. The system includes first electrodes and second electrodes adapted to be implanted within a person's skull. The first electrodes are adapted to be positioned proximate first nerve tissue in the brain associated with a first condition in the person's body for delivering electrical stimulation pulses to the first nerve tissue to treat the first condition. The second electrodes are adapted to be positioned proximate second nerve tissue in the brain associated with a second condition in the person's body for delivering electrical stimulation pulses to the second nerve tissue to treat the second condition. The system also includes a stimulation source operable to generate the first electrical stimulation pulses according to one or more first stimulation programs for delivery to the first nerve tissue and to generate the second electrical stimulation pulses according to one or more second stimulation programs for delivery to the second nerve tissue.

### RELATED APPLICATIONS

This application is a continuation-in-part of and/or claims the benefit of the following co-pending and commonly assigned applications: (i) U.S. Provisional Application Ser. No. 60/645,405, filed Jan. 19, 2005, entitled “Electrical Stimulation System and Method for Stimulating Multiple locations of Target Nerve Tissue in the Brain to Treat Multiple Conditions in the Body;” (ii) U.S. patent application Ser. No. 10/994,008, filed Nov. 18, 2004, entitled “Electrical Stimulation System, Lead, and Method Providing Reduced Neuroplasticity Effects,” (which in turn claims the benefit of U.S. Provisional Application Ser. No. 60/523,710, filed Nov. 20, 2003, entitled “Electrical Stimulation System, Lead, and Method Providing Reduced Neuroplasticity Effects;”); (iii) U.S. patent application Ser. No. 11/207,086, filed Aug. 18, 2005, entitled “Electrical Stimulation System, Lead, and Method Providing Modified Reduced Neuroplasticity Effect,” (which in turn claims the benefit of U.S. Provisional Application Ser. No. 60/602,522, filed Aug. 18, 2004, entitled “Electrical Stimulation System, Lead, and Method Providing Modified Neuroplasticity Effects in a Person's Nerve Tissue”), all of which are incorporated herein by reference

### TECHNICAL FIELD OF THE INVENTION

This invention relates generally to electrical stimulation of a person's brain and in particular to an electrical stimulation system and method for stimulating multiple locations of target nerve tissue in the brain to treat multiple conditions in the body.

### BACKGROUND

Many people experience adverse conditions associated with functions of the cortex, the thalamus, and other brain structures. Such conditions have been treated effectively by delivering electrical energy to one or more target areas of the brain. One method of delivering electrical energy to the brain involves inserting an electrical stimulation lead through a burr hole formed in the skull and then positioning the lead in a precise location proximate a target area of the brain to be stimulated such that stimulation of the target area causes a desired clinical effect. For example, one desired clinical effect may be cessation of tremor from a movement disorder such as Parkinson's Disease. A variety of other clinical conditions may also be treated with deep brain stimulation, such as essential tremor, tremor from multiple sclerosis or brain injury, or dystonia or other movement disorders. The electrical stimulation lead implanted in the brain is connected to an electrical signal generator implanted at a separate site in the body, such as in the upper chest.

Electrical stimulation may also be applied to nerve tissue in the spinal cord or a peripheral nerve to treat regions of the body affected by chronic pain from a variety of etiologies. According to one technique, a set of efficacious stimulation parameters are determined, the set of parameters is entered into a stimulation system, and the stimulation system is used to electrically stimulate particular nerve tissue in the spinal cord or a peripheral nerve according to the set of parameters. Typically, an implanted pulse generator transmits a pulse of electrical energy to an implanted electrical stimulation lead according to the set of parameters and, in response to the pulse, the electrodes of the implanted stimulation lead deliver the electrical energy to the particular nerve tissue in the spinal cord or a peripheral nerve. The electrical energy stimulates the particular nerve tissue in the spinal cord or a peripheral nerve to cause a subjective sensation of numbness or tingling in the affected region of the body, known as “paresthesia,” which masks or otherwise relieves pain in the affected region. For example, the electrodes may be located external to the dura adjacent particular nerve tissue in the spinal cord that is to be stimulated. The location of the electrodes typically must be precisely determined based on the location of the particular nerve tissue in the spinal cord or a peripheral nerve.

### SUMMARY OF THE INVENTION

The electrical stimulation system and method of the present invention may reduce or eliminate certain problems and disadvantages associated with previous techniques for treating conditions in the body.

According to one embodiment, an electrical stimulation system is provided for electrically multiple locations of target nerve tissue in a person's brain to treat multiple conditions in the person's body. The system includes one or more first electrodes and one or more second electrodes adapted for implantation inside a person's skull. The one or more first electrodes are adapted to be positioned proximate first target nerve tissue in the brain associated with a first condition in the person's body and to deliver first electrical stimulation pulses to the first target nerve tissue according to one or more first stimulation programs to treat the first condition in the person's body. The one or more second electrodes are adapted to be positioned proximate second target nerve tissue in the brain associated with a second condition in the person's body and to deliver second electrical stimulation pulses to the second target nerve tissue according to one or more second stimulation programs to treat the second condition in the person's body. The system also includes a stimulation source operable to generate (a) the first electrical stimulation pulses according to the one or more first stimulation programs for transmission to the one or more first electrodes to cause the one or more first electrodes to deliver the first electrical stimulation pulses to the first target nerve tissue to treat the first condition in the person's body, and (b) the second electrical stimulation pulses according to the one or more second stimulation programs for transmission to the one or more second electrodes to cause the one or more second electrodes to deliver the second electrical stimulation pulses to the second target nerve tissue to treat the second condition in the person's body.

According to one embodiment, a method is provided for electrically multiple locations of target nerve tissue in a person's brain to treat multiple conditions in the person's body. A stimulation source is used to generate first electrical stimulation pulses according to one or more first stimulation programs for transmission to a plurality of first electrodes implanted inside the person's skull to cause the first electrodes to deliver the first electrical stimulation pulses to the first target nerve tissue in the person's brain associated with a first condition in the person's body. The stimulation source is also used to generate second electrical stimulation pulses according to one or more second stimulation programs for transmission to a plurality of second electrodes implanted inside the person's skull to cause the second electrodes to deliver the second electrical stimulation pulses to the second target nerve tissue in the person's brain associated with a second condition in the person's body. In response to receiving the first electrical stimulation pulses transmitted from the stimulation source, the one or more first implanted electrodes deliver the first electrical stimulation pulses to the first target nerve tissue in the brain according to the one or more first stimulation programs to treat the first condition in the person's body. Similarly, in response to receiving the second electrical stimulation pulses transmitted from the stimulation source, the one or more second implanted electrodes deliver the second electrical stimulation pulses to the second target nerve tissue in the brain according to the one or more second stimulation programs to treat the second condition in the person's body.

Particular embodiments of the present invention may provide one or more technical advantages. According to the present invention, an electrical stimulation system is used to provide therapeutic electrical stimulation to multiple locations of target nerve tissue in a person's brain to treat multiple conditions in the person's body. The electrical stimulation system may include one or more electrical stimulation leads each having one or more electrodes. The one or more leads are implanted inside a person's skull such that a first set of electrodes are located proximate a first location of target nerve tissue in the brain corresponding to a first condition in the person's body and a second set of electrodes are located proximate a second location of target nerve tissue in the brain corresponding to a second condition in the person's body. The first and second sets of electrodes deliver electrical stimulation pulses to the first and second locations of target nerve tissue, respectively, which partially or completely alleviates the first and second conditions in the person's body, which may significantly increase the person's quality of life. In this manner, multiple conditions in a person's body may be treated using a single stimulation system having a single stimulation source, such as a single implantable pulse generator for example, rather than requiring multiple stimulation systems and/or multiple stimulation sources.

The first and second sets of electrodes may deliver such electrical stimulation at least substantially simultaneously, in an alternating or multiplexed manner, or in any other suitable manner. The one or more electrical stimulation leads may be precisely positioned using a neuronavigation system that includes brain imaging information and mapping data obtained from the imaging of the person's brain or from the imaging of the brains of one or more other patients. In addition, non-invasive transcranial magnetic stimulation (TMS) of the target nerve tissue may be performed before surgically implanting the electrical stimulation lead in order to determine whether the person is a candidate for receiving an implanted electrical stimulation system.

Certain embodiments may provide all, some, or none of these advantages. Certain embodiments may provide one or more other advantages, one or more of which may be apparent to those skilled in the art from the figures, descriptions, and claims included herein.

### BRIEF DESCRIPTION OF THE DRAWINGS

For a more complete understanding of the present invention and advantages thereof, reference is now made to the following description taken in conjunction with the accompanying drawings, in which:

FIGS. 1A-1B illustrate example electrical stimulation systems for electrically stimulating multiple locations of target nerve tissue in the brain to treat multiple conditions in the body;

FIGS. 2A-2H illustrate example pairs of electrical stimulation leads that may be used for electrically stimulating multiple locations of target nerve tissue in the brain to treat multiple conditions in the body;

FIGS. 3A-3H illustrate example electrical stimulation leads having multiple sets of electrodes for electrically stimulating multiple locations of target nerve tissue in the brain to treat multiple conditions in the body;

FIG. 4 illustrates example placement of the electrical stimulation system shown in FIGS. 1A-1B within a person's body;

FIG. 5 is a cross-section of a portion of the person's head shown in FIG. 4, illustrating example locations of multiple electrical stimulation leads;

FIG. 6 illustrates an example method for determining an optimal location and implanting the stimulation system of FIGS. 1A-1B into a person in order to electrically stimulate multiple locations of target nerve tissue in the brain identified through imaging of the brain to treat multiple conditions in the body;

FIG. 7 illustrates an example stimulation set;

FIG. 8 illustrates a number of example stimulation programs, each of which includes a number of stimulation sets;

FIG. 9 illustrates example execution of a sequence of stimulation sets within an example stimulation program;

FIG. 10 illustrates an example of a stimulation set of a stimulation program for a first set of electrodes being executed at least substantially simultaneously with a stimulation set of a stimulation program for a second set of electrodes according to one embodiment of the invention;

FIG. 11 illustrates an example of a stimulation set of a stimulation program for a first set of electrodes being executed in an alternating, or multiplexed, manner with a stimulation set of a stimulation program for a second set of electrodes according to one embodiment of the invention; and

FIG. 12 illustrates an example of multiple stimulation sets of a first stimulation program for a first set of electrodes being executed in an alternating, or multiplexed, manner with multiple stimulation sets of a second stimulation program for a second set of electrodes according to one embodiment of the invention.

### DESCRIPTION OF EXAMPLE EMBODIMENTS

According to the present invention, an electrical stimulation system is used to electrically stimulate multiple locations of target nerve tissue in a person's brain to treat multiple conditions in the body.

In particular, multiple sets of electrodes located on one or more electrical stimulation leads are implanted inside a person's skull such that the multiple sets of electrodes are located proximate different target nerve tissue at different locations in the brain. For example, a first set of one or more electrodes may be positioned proximate first target nerve tissue at a first location in the brain and a second set of one or more electrodes may be positioned proximate second target nerve tissue at a second location in the brain. As used herein, the term “proximate” means on, in, adjacent, or near. Thus, each set of one or more electrodes is adapted to be positioned on, in, adjacent, or near target nerve tissue in the brain corresponding with that set of electrodes. In addition, the term “nerve tissue in the brain” as used herein includes any neural tissue in any neural region of the brain, including gray matter and white matter that make up the brain.

The multiple sets of electrodes may be located on a single stimulation lead coupled to a single stimulation source or, alternatively, each set of electrodes may be located on a separate stimulation lead coupled to the single stimulation source. Thus, for example, a first set of electrodes may be located on a first stimulation lead coupled to a single stimulation source and a second set of electrodes may be located on a second stimulation lead coupled to the single stimulation source.

Each location of nerve tissue in the brain to be stimulated by a set of electrodes may be identified by a notable level of activity, such as overactivity or underactivity for example, associated with a condition in the person's body, such as pain in a region of the person's body or tinnitus for example. Each set of electrodes may deliver electrical stimulation pulses to an identified location of target nerve tissue in the brain to adjust the level of activity in that location of target nerve tissue to treat the condition in the person's body corresponding with that location of target nerve tissue. For example, if the identified location of target nerve tissue in the brain is overactive, the one or more electrodes may deliver appropriate electrical stimulation pulses to decrease the activity of the identified location of target nerve tissue to treat the condition in the person's body. Similarly, if the identified location of target nerve tissue in the brain is underactive, the one or more electrodes may deliver appropriate electrical stimulation pulses to increase the activity of the identified location of target nerve tissue to treat the condition in the person's body.

By providing an electrical stimulation system having multiple sets of electrodes, each set of electrodes may be used to adjust the level of activity in a particular location of target nerve tissue to treat the condition in the person's body corresponding with that location of target nerve tissue. Thus, multiple conditions in the person's body may be treated using a single electrical stimulation having a single stimulation pulse generator. For example, using an electrical stimulation system having two sets of electrodes, the first set of electrodes may deliver appropriate electrical stimulation pulses to decrease the activity of overactive brain tissue in a first location in the brain, and the second set of electrodes may deliver appropriate electrical stimulation pulses to decrease the activity of overactive brain tissue in a second location in the brain. As another example, the first set of electrodes may deliver appropriate electrical stimulation pulses to increase the activity of underactive brain tissue in a first location in the brain, and the second set of electrodes may deliver appropriate electrical stimulation pulses to increase the activity of underactive brain tissue in a second location in the brain. As yet another example, the first set of electrodes may deliver appropriate electrical stimulation pulses to increase the activity of underactive brain tissue in a first location in the brain, and the second set of electrodes may deliver appropriate electrical stimulation pulses to decrease the activity of overactive brain tissue in a second location in the brain.

The conditions in the person's body may include any conditions associated with a notable level of activity, such as overactivity or underactivity for example, in one or more locations of nerve tissue in the person's brain. Example conditions may include pain in a region of the person's body, tinnitus, depression, and other neurological, physiological, psychological, or other disorders. In some instances, the notable level of activity in a location of target nerve tissue in the person's brain, and thus the condition in the person's body, is caused by damaged, altered, or otherwise abnormally functioning nerve tissue in the person's body correlating to the location of target nerve tissue in the person's brain. For example, with respect to pain, damaged, altered, or otherwise abnormally functioning nerve tissue in a region of a person's body that causes pain in that region or another region of the person's body may cause overactivity or underactivity in nerve tissue in the person's brain that correlates to the abnormally functioning nerve tissue. As another example, with respect to tinnitus, damaged, altered, or otherwise abnormally functioning nerve tissue in a person's auditory system or brain that causes tinnitus may cause overactivity or underactivity in nerve tissue in the person's brain that correlates to the abnormally functioning nerve tissue.

Each set of stimulation electrodes may be precisely positioned using a neuronavigation system that includes brain imaging and mapping data received from the imaging of the person's brain. In addition, non-invasive transcranial magnetic stimulation (TMS) of the person's brain may be performed before surgically implanting the one or more electrical stimulation leads on which the sets of electrodes are located in order to determine whether the person is a candidate for receiving an implanted electrical stimulation system.

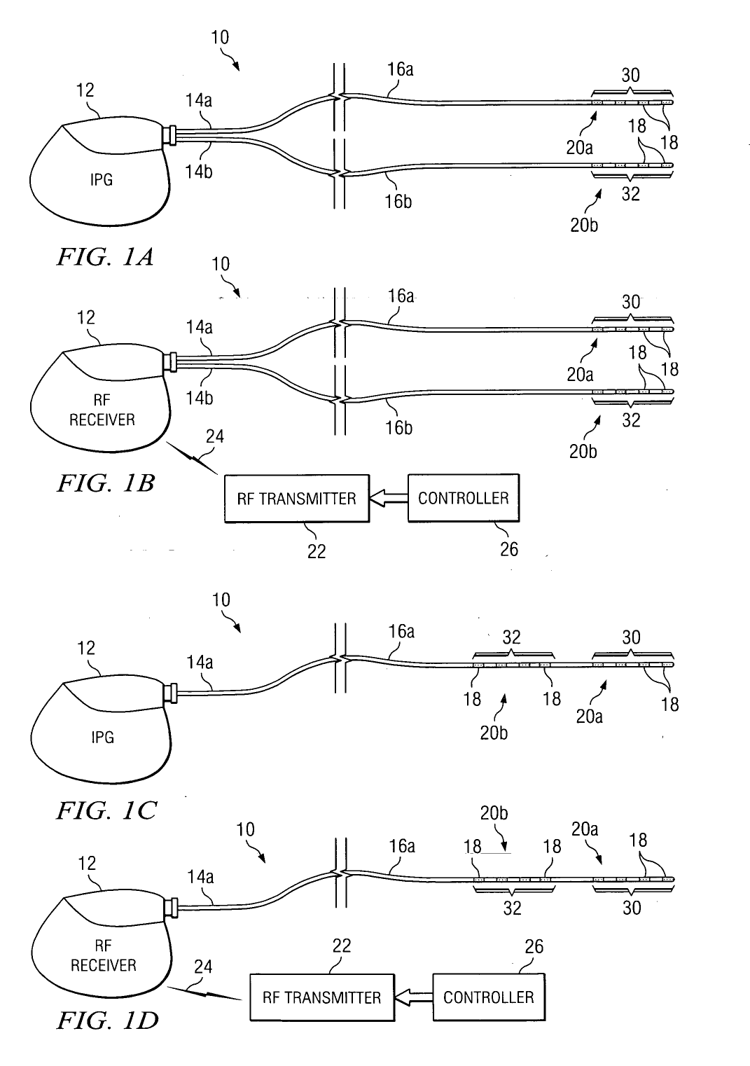

FIGS. 1A-1D illustrate example electrical stimulation systems 10 for electrically stimulating multiple locations of target nerve tissue in the brain to treat multiple conditions in the body. In some embodiments, each location of target nerve tissue in the brain to be stimulated may be identified through imaging of the person's brain as having a notable level of activity corresponding to a condition in the person's body. Thus, stimulation system 10 may generate and apply a stimulus to each of multiple locations of target nerve tissue in a person's brain identified through imaging of the person's brain as having a notable level of activity to adjust the level of activity in each of the identified locations of target nerve tissue to treat multiple conditions in the person's body corresponding to the identified locations of target nerve tissue.

In general terms, stimulation system 10 includes an implantable electrical stimulation source 12 and one or more implantable electrical stimulation leads 14 for applying electrical stimulation pulses to multiple locations of nerve tissue in the brain. In operation, the electrical stimulation source 12 and the one or more stimulation leads 14 are implanted in the person's body, as discussed below with reference to FIG. 4. FIGS. 1A and 1B illustrate example stimulation systems 10 that include an implantable electrical stimulation source 12 and two implantable stimulation leads 14a and 14b, whereas FIGS. 1C and 1D illustrate example stimulation system 10 that include an implantable electrical stimulation source 12 and a single implantable stimulation lead 14.

Stimulation source 12 is coupled to a connecting portion 16 of stimulation lead 14. In certain embodiments, stimulation source 12 is physically coupled to connecting portion 16 of stimulation lead 14. In certain other embodiments, stimulation source 12 is coupled to stimulation lead 14 via a wireless link enabling wireless communications between stimulation source 12 and stimulation lead 14. For example, such a stimulation lead 14 may be a Bion® stimulation lead manufactured by Advanced Bionics Corporation. Whether stimulation source 12 is coupled physically or wirelessly to stimulation lead 14, stimulation source 12 controls the stimulation pulses transmitted to stimulation electrodes 18 located on stimulation lead 14, as discussed below.

Stimulation source 12 controls the electrical stimulation pulses transmitted to each of multiple sets of one or more electrodes 18 located on one or more stimulation leads 14. For example, as shown in FIGS. 1A-1B, a first set 30 of electrodes 18 is located on a stimulating portion 20a of stimulation lead 14a, and a second set 32 of electrodes 18 is located on a stimulating portion 20b of stimulation lead 14b. As another example, as shown in FIGS. 1C-1D, a first set 30 of electrodes 18 is located on a stimulating portion 20 of a single stimulation lead 14a and a second set 32 of electrodes 18 is located on the stimulating portion 20 of the single stimulation lead 14a. First set 30 of electrodes 18 may be located proximate a first location of target nerve tissue and may deliver electrical stimulation pulses received from stimulation source 12 to the first location of target nerve tissue, and second set 32 of electrodes 18 may be simultaneously located proximate a second location of target nerve tissue and may deliver electrical stimulation pulses received from stimulation source 12 to the second location of target nerve tissue.

As discussed below in greater detail, stimulation source 12 may generate electrical stimulation pulses for first set 30 and second set 32 of electrodes 18 according to a plurality of stimulation programs, each stimulation program including one or more stimulation sets, each stimulation set specifying suitable stimulation parameters (e.g., amplitude or intensity information, frequency information, phase information, pulse width information, etc.) for electrical stimulation pulses. Thus, as shown in FIGS. 1A-1D, stimulation source 12 may generate (a) electrical stimulation pulses for first set 30 of electrodes 18 according to one or more first stimulation programs and (b) electrical stimulation pulses for second set 32 of electrodes 18 according to one or more second stimulation programs, which may or may not be different than the one or more first stimulation programs. As discussed below with reference to FIGS. 10-12, stimulation source 12 may generate and transmit electrical stimulation pulses to first set 30 of electrodes 18 and second set 32 of electrodes 18 substantially simultaneously, in an alternating or multiplexed manner, or in any other suitable manner. A doctor, the patient, or another user of stimulation source 12 may directly or indirectly input and/or control the stimulation programs or particular stimulation parameters to specify and/or modify the nature of the electrical stimulation provided to one or more sets of electrodes 18 (such as first set 30 and/or second set 32, for example).

In certain embodiments, as shown in FIGS. 1A and 1C, stimulation source 12 includes an implantable pulse generator (IPG). An example IPG may be one manufactured by Advanced Neuromodulation Systems, Inc., such as the Genesis® System, part numbers 3604, 3608, 3609, and 3644. In certain other embodiments, as shown in FIGS. 1B and 1D, stimulation source 12 includes an implantable wireless receiver. An example wireless receiver may be one manufactured by Advanced Neuromodulation Systems, Inc., such as the Renew® System, part numbers 3408 and 3416. The wireless receiver is capable of receiving wireless signals from a wireless transmitter 22 located external to the person's body. The wireless signals are represented in FIGS. 1B and 1D by wireless link symbol 24. A doctor, the patient, or another user of stimulation source 12 may use a controller 26 located external to the person's body to provide control signals for operation of stimulation source 12. Controller 26 provides the control signals to wireless transmitter 22, wireless transmitter 22 transmits the control signals and power to the wireless receiver of stimulation source 12, and stimulation source 12 uses the control signals to vary the stimulation parameters of electrical stimulation pulses transmitted through stimulation leads 14a and 14b to the stimulation sites. An example wireless transmitter 22 may be one manufactured by Advanced Neuromodulation Systems, Inc., such as the Renew® System, part numbers 3508 and 3516.

Although each embodiment of stimulation system 10 discussed herein includes a stimulation source 12 operable to generate stimulation pulses for either a pair of stimulation leads 14a, 14b each including a single set 30, 32 of electrodes 18 (for example, see FIGS. 1A-1B), or for a single stimulation lead 14 including two sets 30, 32 of electrodes 18 (for example, see FIGS. 1C-1D), in other embodiments of stimulation system 10 may include a stimulation source 12 operable to generate electrical stimulation pulses for any suitable number of sets of electrodes 18 located on any suitable number of stimulation leads 14 in order to treat any number of conditions in a person's body. For example, a stimulation system 10 may include a stimulation source 12 operable to generate electrical stimulation pulses for three sets of electrodes 18, each located on a separate stimulation lead 14, for stimulating three different locations of target nerve tissue in a person's brain to treat three conditions in a person's body. As another example, a stimulation system 10 may include a stimulation source 12 operable to generate electrical stimulation pulses for three sets of electrodes 18 located on a single stimulation lead 14 for stimulating three different locations of target nerve tissue in a person's brain to treat three conditions in a person's body.

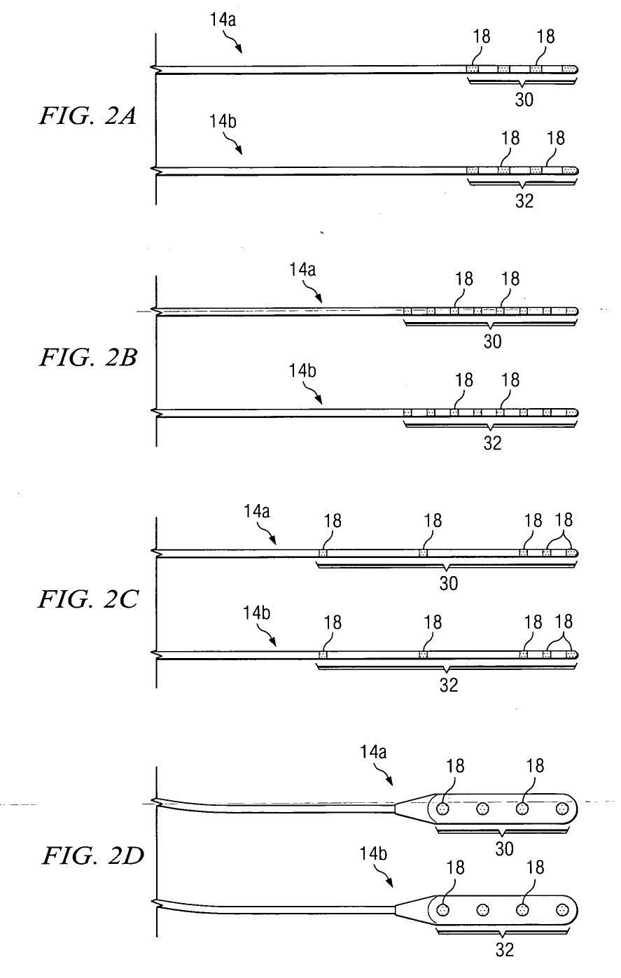

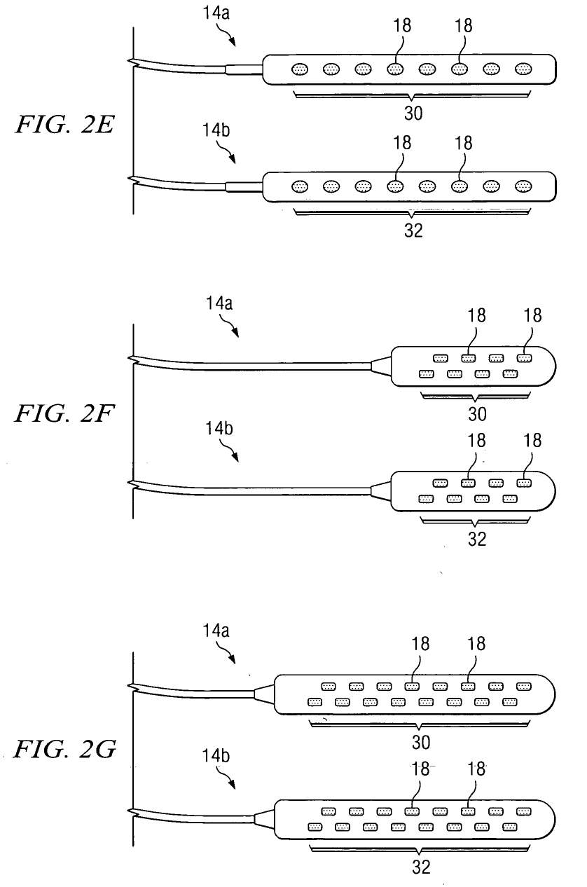

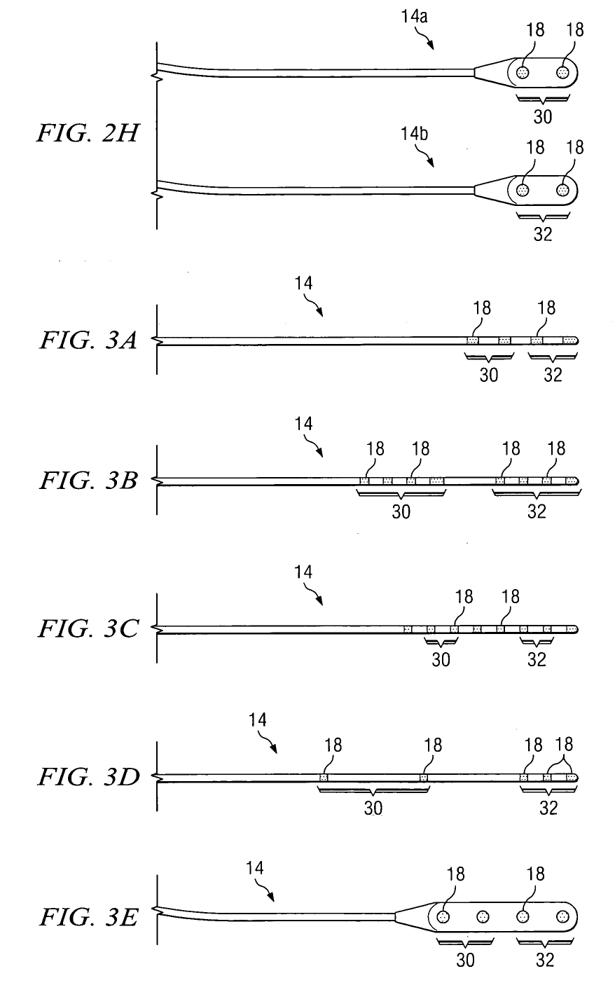

FIGS. 2A-2H illustrate example pairs of electrical stimulation leads 14a and 14b that may be used in electrical stimulation system 10 for electrically stimulating multiple locations of target nerve tissue in the brain to treat multiple conditions in the body. In each example, the first set 30 of electrodes is located on lead 14a, and the second set 32 of electrodes is located on lead 14b. As discussed above, the first set 30 of electrodes 18 is adapted to be positioned proximate a first location of target nerve tissue in a person's brain and used to deliver to the first location of target nerve tissue electrical stimulation pulses received from stimulation source 12 according to one or more first stimulation programs. The second set 32 of electrodes 18 is adapted to be positioned proximate a second location of target nerve tissue in a person's brain and used to deliver to the second location of target nerve tissue electrical stimulation pulses received from stimulation source 12 according to one or more second stimulation programs.

A percutaneous lead 14, such as example leads 14a and 14b shown in FIGS. 2A-2C, includes one or more circumferential electrodes 18 spaced apart from one another along the length of the lead 14. Circumferential electrodes 18 emit electrical stimulation energy generally radially in all directions. A laminotomy, paddle, or surgical lead 14, such as example leads 14a and 14b shown in FIGS. 2D-2H, includes one or more directional electrodes 18 spaced apart from one another along one surface of the lead 14. Directional electrodes 18 emit electrical stimulation energy in a direction generally perpendicular to the surface of the lead 14 on which they are located. Although various types of leads 14 are shown as examples, the present invention contemplates stimulation system 10 including any suitable type of leads in any suitable number. In addition, although FIGS. 2A-2H each illustrate an example pair of the same type of leads 14a and 14b, any number and types of leads 14 may be used together in an electrical stimulation system 10. For example, an electrical stimulation system 10 may include a pair of leads 14 including a first lead 14a of the type shown in FIG. 2A and second lead 14b of the type shown in FIG. 2D.

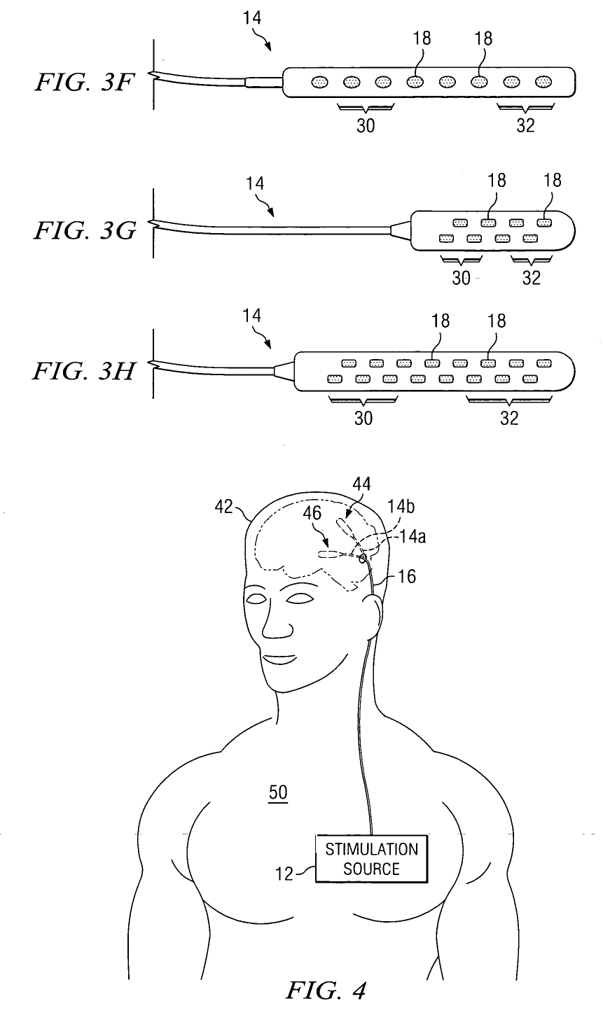

FIGS. 3A-3H illustrate example electrical stimulation leads 14 having multiple sets of electrodes 18 for electrically stimulating multiple locations of target nerve tissue in the brain to treat multiple conditions in the body. Example leads 14 shown in FIGS. 3A-3D are percutaneous leads, as discussed above with reference to FIGS. 2A-2C. Example leads 14 shown in FIGS. 3E-3H are laminotomy, paddle, or surgical leads, as discussed above with reference to FIGS. 2D-2H. Each lead 14 includes a first set 30 of electrodes 18 and a second set 32 of electrodes 18. The first set 30 of electrodes 18 is adapted to be positioned proximate a first location of target nerve tissue in a person's brain and used to deliver to the first location of target nerve tissue electrical stimulation pulses received from stimulation source 12 according to one or more first stimulation programs, and the second set 32 of electrodes 18 is adapted to be positioned proximate a second location of target nerve tissue in a person's brain and used to deliver to the second location of target nerve tissue electrical stimulation pulses received from stimulation source 12 according to one or more second stimulation programs.

In some embodiments, the first set 30 of electrodes 18 on a particular lead 14 comprises a predetermined set of electrodes 18, and the second set 32 of electrodes 18 on the particular lead 14 comprises another predetermined set of electrodes 18. Thus, each electrode 18 on a particular lead 14 may be pre-assigned to the first set 30 of electrodes 18, the second set 32 of electrodes 18, or neither. In other embodiments, one or more electrodes 18 on a particular lead 14 are not pre-assigned, such that they may be dynamically assigned to the first set 30 of electrodes 18, the second set 32 of electrodes 18, or neither. Thus, the electrodes 18 in the first set 30 of electrodes 18 and/or the second set 32 of electrodes 18 may not be predetermined, and may be assigned and/or reassigned as appropriate. For example, suppose lead 14 shown in FIG. 3H is implanted inside the skull and positioned proximate two locations of nerve tissue to be stimulated. The individual electrodes 18 of lead 14 may be assigned to a first set 30 of electrodes 18, a second set 32 of electrodes 18, or neither based on positioning of the lead 14 relative to the two locations of nerve tissue to be stimulated. In particular, one or more electrodes 18 on lead 14 that are positioned proximate the first location of nerve tissue to be stimulated may be assigned to the first set 30 of electrodes 18, and one or more other electrodes 18 on lead 14 that are positioned proximate the second location of nerve tissue to be stimulated may be assigned to the second set 32 of electrodes 18.

FIG. 4 illustrates example placement of the electrical stimulation system 10 shown in FIGS. 1A-1B within a person's body. Stimulation leads 14a and 14b are implanted inside the person's skull 42 proximate a particular region of the person's brain. In certain embodiments, one or both of stimulation leads 14a and 14b are positioned within the extradural region adjacent the brain such that the first set 30 of electrodes 18 and/or the second set 32 of electrodes 18 are located proximate target nerve tissue within one or more regions of the brain, for example, the frontal lobe, the occipital lobe, the parietal lobe, the temporal lobe, the cerebellum, or the brain stem. In other embodiments, one or both of stimulation leads 14a and 14b are positioned within the brain for providing deep brain stimulation, such as for treating conditions such as essential tremor, tremor from multiple sclerosis or brain injury, or dystonia or other movement disorders, for example.

As shown in FIG. 4, stimulation lead 14a may be positioned such that the first set 30 of electrodes 18 are positioned proximate a first location of target nerve tissue 44 corresponding to a first condition in the person's body, and stimulation lead 14b may be positioned such that the second set 32 of electrodes 18 are positioned proximate a second location of target nerve tissue 46 corresponding to a second condition in the person's body.

As an example, in certain embodiments in which electrical stimulation system 10 is used to provide pain relief to one or more painful regions of a person's body, perhaps in addition to treating one or more other conditions in the person's body, target nerve tissue 44 and/or 46 in the brain may be located in one or more of the primary somatosensory cortex, the secondary somatosensory cortex, the putamen, the thalamus, the insula, the anterior cingulate cortex, the supplementary motor area, and the frontal operculum. As another example, in certain embodiments in which electrical stimulation system 10 is used to reduce tinnitus, perhaps in addition to treating one or more other conditions in the person's body, target nerve tissue 44 and/or 46 in the brain may be located in an area of cortical reorganization in the temporal lobe of the person's brain that causes the tinnitus. In some instances, with respect to tinnitus, the area of cortical reorganization, and thus target nerve tissue 44 and/or 46, is located in one or more locations in the auditory cortex, such as the primary auditory cortex, AI, also known as the transverse temporal gyri of Heschl (Brodman's areas 41 and 42), the secondary auditory cortex, AII (Brodman's areas 22 and 52), the remote projection region, the ventral medial geniculate, which projects almost entirely to AI, the surrounding auditory areas, which receive projections from the rest of the geniculate body, and the medial geniculate body, which is the major auditory nucleus of the thalamus. In certain embodiments, one or both of stimulation leads 14a and 14b are located at least partially within or below the dura mater proximate target nerve tissue 44 and/or 46. For example, one or both of stimulation leads 14a and 14b may be inserted into the cortex or deeper layers of the brain.

Target nerve tissue 44 and target nerve tissue 46 stimulated by first set 30 of electrodes 18 and second set 32 of electrodes 18, respectively, may be located at any distance from each other, and may or may not be located in the same region (such as the same lobe, cortex, or area) of the brain. For example, target nerve tissue 44 and target nerve tissue 46 may be located in the same one, or in different ones, of the following regions: the frontal lobe, the occipital lobe, the parietal lobe, the temporal lobe, the cerebellum, and the brain stem. As another example, target nerve tissue 44 and target nerve tissue 46 may be located in the same one, or in different ones, of the following regions: the primary auditory cortex, the secondary auditory cortex, the remote projection region, Brodman's area 41, Brodman's area 42, Brodman's area 22, and Brodman's area 52. The conditions in the person's body corresponding to target nerve tissue 44 and target nerve tissue 46 may be substantially related, substantially unrelated, or completely unrelated.

Stimulation source 12 is implanted within a subcutaneous pocket within the person's torso 50 (such as in or near the chest area or buttocks), and connecting portion 16 (which in some embodiments includes connecting portions 16a and 16b) is tunneled, at least in part, subcutaneously underneath the person's skin to connect stimulation source 12 with the stimulation leads 14a and 14b. However, stimulation source 12 may be located at any suitable location within the person's body according to particular needs.

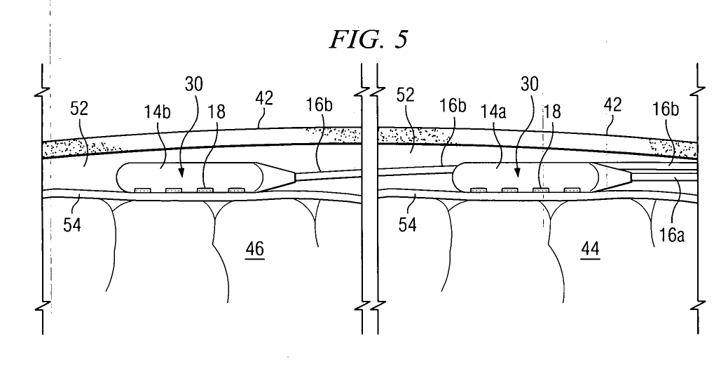

FIG. 5 is a cross-section of a portion of the person' head shown in FIG. 4, illustrating example locations of electrical stimulation leads 14a and 14b. In certain embodiments, as discussed above, stimulation lead 14a and 14b are located in the extradural region 52 outside the dura mater 54 and proximate target nerve tissue 44 and 46, respectively, within the brain.

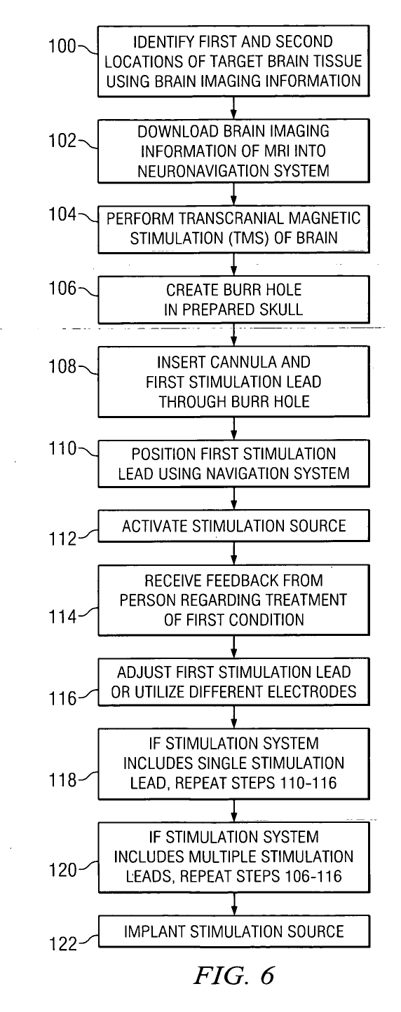

FIG. 6 illustrates an example method for determining an optimal location and implanting the stimulation system 10 of FIGS. 1A-1B into a person in order to electrically stimulate target nerve tissue in the person's brain identified through imaging of the brain to treat multiple conditions in the body. At step 100, at least a portion of the person's brain may be imaged and/or mapped using one or more imaging techniques to obtain brain imaging information and to identify a first location of target nerve tissue 44 and a second location of target nerve tissue 46 in the brain having a notable level of activity, such as overactivity or underactivity for example, which could be associated with multiple conditions in the person's body. The brain imaging information may also indicate whether each identified location of target nerve tissue 44 and 46 is overactive or underactive and the degree or intensity of such overactivity or underactivity. Imaging techniques used to obtain such brain imaging information may include, for example, positron emission tomography (PET), magnetic resonance imaging (MRI), functional MRI (fMRI), electroencephalography (EEG), magnetoencephalography (MEG), x-ray computed tomography (CT), single photon emission computed tomography (SPECT), brain electrical activity mapping (BEAM), transcranial magnetic stimulation (TMS), electrical impedance tomography (EIT), near-infrared spectroscopy (NIRS), and optical imaging.

The techniques discussed above may be used as confirmation or investigation of the notable level of activity of the target nerve tissue. Additionally, the first location of target nerve tissue 44 and/or the second location of target nerve tissue 46 in the person's brain may be determined using information from brain imaging studies performed on other patients, and thus the imaging of the person's brain at step 100 may not be performed. Such brain imaging studies may include imaging information obtained using one or more of the imaging techniques listed above to image the brains of patients suffering from various types of conditions. The first location of target nerve tissue 44 and/or the second location of target nerve tissue 46 in the brain may be identified using statistical analysis of such imaging information. Thus, at step 100, first location of target nerve tissue 44 corresponding to a first condition in the person's body and/or second location of target nerve tissue 46 corresponding to a second condition in the person's body may be identified according to the results of such brain imaging studies.

At step 102, the brain imaging information obtained at step 100 (whether from imaging the person's brain or from imaging studies of other patients suffering from the same or similar condition as the person) is downloaded into a neuronavigation system. At step 104, TMS of one or more areas of the person's brain, such as areas including the locations of target nerve tissue 44, 46 identified at step 100 for example, may be performed to determine whether the person is a candidate for receiving an implanted electrical stimulation system 10. The TMS process, which is a non-invasive technique of activating or deactivating focal areas of the brain, may be guided by the navigation system that includes the brain imaging information obtained at step 100. If the TMS process is successful in treating the conditions in the person's body corresponding to the locations of target nerve tissue 44, 46 identified at step 100, the person may be considered a candidate for receiving an implanted electrical stimulation system 10.

Electrical stimulation system 10 is implanted inside the person at steps 106 through 132. Stimulation lead 14a is implanted at steps 106-110. At step 106, the skull 42 is first prepared by exposing the skull 42 and creating a burr hole or other opening in the skull 42. A burr hole cover may be seated within the burr hole and fixed to the scalp or skull 42. Stereotactic equipment suitable to aid in placement of one or more stimulation leads 14 in the brain may be positioned around the head. An insertion cannula for a stimulation lead may be inserted through the burr hole into the brain at step 108, but a cannula is not typically used where stimulation lead 14 is a laminotomy, paddle, or surgical lead. A cannula and stimulation lead 14a may be inserted together or stimulation lead 14a may be inserted through the cannula after the cannula has been inserted. Guided by the navigation system that includes the brain imaging information obtained at step 100, stimulation lead 14a is precisely positioned proximate the brain at step 110 such that the first set 30 of electrodes 18 are located proximate the first location of target nerve tissue 44 in the brain identified at step 100. In certain embodiments, stimulation lead 14 is positioned extradurally, such as shown in FIG. 4.

At step 112, stimulation source 12 is activated to generate and send electrical stimulation pulses via the first set 30 of electrodes 18 to the first location of target nerve tissue 44. The electrical stimulation pulses delivered to the first target nerve tissue 44 by the first set 30 of electrodes 18 may adjust the activity of first target nerve tissue 44 in an appropriate manner to treat the first condition in the person's body corresponding to the first target nerve tissue 44. For example, if the brain imaging information obtained at step 100 indicates that the first location of the first target nerve tissue 44 is overactive, stimulation source 12 may generate, and the first set 30 of electrodes 18 may deliver, appropriate electrical stimulation pulses to decrease the activity of the first target nerve tissue 44 to treat the first condition in the person's body corresponding to the first target nerve tissue 44. Similarly, if the brain imaging information obtained at step 100 indicates that the first target nerve tissue 44 is underactive, stimulation source 12 may generate, and the first set 30 of electrodes 18 may deliver, appropriate electrical stimulation pulses to increase the activity of the first target nerve tissue 44 to treat the first condition in the person's body.

At step 114, the person indicates whether the first condition in the person's body corresponding with the first target nerve tissue 44 is adequately alleviated by electrical stimulation delivered by the first set 30 of electrodes 18. If the first condition is not adequately alleviated, stimulation lead 14a may be moved incrementally, different electrodes 18 within first set 30 may be utilized, or both at step 116 until the person indicates that the first condition is adequately alleviated.

As indicated at step 118, the stimulation system 10 being implanted includes a single stimulation lead 14a including both first set 30 and second set 32 of electrodes 18, steps 110-116 are also performed with respect to second set 32 of electrodes 18 to properly position second set 32 of electrodes 18 proximate the second location of target nerve tissue 46 such that the second condition in the person's body corresponding with the second target nerve tissue 46 is adequately alleviated by electrical stimulation delivered by the second set 32 of electrodes 18. The proper positioning of first set 30 of electrodes 18 proximate the first target nerve tissue 44 and the proper positioning of second set 32 of electrodes 18 proximate the second target nerve tissue 46 may be performed sequentially in a substantially independent manner, or concurrently in a substantially interdependent manner. Thus, any two or more of the steps discussed above may be performed substantially simultaneously or in an otherwise interdependent manner in order to properly position first set 30 and second set 32 of electrodes 18 proximate first target nerve tissue 44 and second target nerve tissue, respectively. Once stimulation lead 14a has been properly positioned in the brain such that the first and second conditions in the person's body are adequately alleviated, stimulation lead 14a may be uncoupled from any stereotactic equipment present, which may then be removed.

Alternatively, as indicated at step 120, if stimulation system 10 being implanted includes multiple leads 14a and 14b, steps 106-116 are also performed for stimulation lead 14b to implant and position stimulation lead 14b inside the skull 42 such that the second set 32 of electrodes 18 on stimulation lead 14b are positioned proximate the second location of target nerve tissue 46 in the brain identified at step 100. A new burr hole may or may not be required at step 106, depending on the relative locations of first target nerve tissue 44 and second target nerve tissue 46. As discussed above with reference to stimulation lead 14a, stimulation lead 14b may be inserted, positioned, and adjusted until the person's condition corresponding with target nerve tissue 46 is adequately alleviated. Stimulation lead 14b may then be secured and uncoupled from any stereotactic equipment, which may then be removed.

Once one or more stimulation leads 14 have been inserted and secured, stimulation source 12 is implanted at step 122. The implant site is typically a subcutaneous pocket formed to receive and house stimulation source 12. The implant site is usually positioned a distance away from the insertion site, such as in or near the chest or buttocks or another place in the torso 50. Connecting portion 16 of each of the one or more stimulation of leads 14 extends from the lead insertion site to the implant site at which stimulation source 12 is implanted. A doctor, the patient, or another user of stimulation source 12 may directly or indirectly input stimulation parameters for controlling the nature of the electrical stimulation provided.

Although example steps are illustrated and described, the present invention contemplates two or more steps taking place substantially simultaneously or in a different order. In addition, the present invention contemplates using methods with additional steps, fewer steps, or different steps, so long as the steps remain appropriate for determining an optimal location and implanting stimulation system 10 of into a person in order to electrically stimulate multiple locations of target nerve tissue 44, 46 in the brain to treat multiple conditions in the body.

During the operation of stimulation system 10 according to a particular set of stimulation parameters, the efficacy of the stimulation associated with the particular set of stimulation parameters may decrease over time due to neuroplasticity of the brain. Neuroplasticity refers to the ability of the brain to dynamically reorganize itself in response to certain stimuli to form new neural connections. This allows the neurons in the brain to compensate for injury or disease and adjust their activity in response to new situations or changes in their environment. With respect to electrical stimulation, the reduction in efficacy due to neuroplasticity can occur after just a few weeks of treatment. In order to regain the same efficacy, a new set of efficacious electrical stimulation parameters must be determined, the new set of parameters must be entered into the system, and the system is again used to electrically stimulate the brain according to the new set of parameters to continue to treat the condition. This may result in the additional time and expense associated with a return visit to the treating physician for determining and entering the new set of parameters. Especially where treatment is to continue over a relatively long period of time, such as months or years, this additional time and expense poses a significant drawback.

Thus, in certain embodiments, in addition to providing therapeutic electrical stimulation to the brain for treating the condition in the person's body, stimulation system 10 may be capable of applying additional electrical stimulation to the brain to reduce neuroplasticity effects associated with the therapeutic electrical stimulation. In one embodiment, the nature of the neuroplasticity reducing electrical stimulation may be varied more or less continually, in a predetermined or randomized manner, to prevent, delay, or otherwise reduce the ability of the brain to adapt to the neuroplasticity reducing electrical stimulation and dynamically reorganize itself accordingly. In a more particular embodiment, the neuroplasticity reducing electrical stimulation may be randomized or otherwise varied about the therapeutic electrical stimulation to achieve this result. In essence, the randomized or otherwise varied neuroplasticity reducing electrical stimulation makes it more difficult for the brain to dynamically reorganize itself to overcome the effects of the therapeutic electrical stimulation.

In certain other embodiments, stimulation system 10 may similarly be capable of applying additional electrical stimulation to the brain to enhance, rather than reduce, neuroplasticity effects associated with the therapeutic electrical stimulation. In one embodiment, the nature of the neuroplasticity enhancing electrical stimulation may controlled in a predetermined non-randomized manner to promote, accelerate, or otherwise enhance the ability of the brain to adapt to the neuroplasticity enhancing electrical stimulation and dynamically reorganize itself accordingly. In essence, the predetermined non-randomized neuroplasticity enhancing electrical stimulation facilitates the brain dynamically reorganizing itself in response to the therapeutic electrical stimulation. Techniques analogous to some or all of those discussed below for reducing neuroplasticity effects may be employed for enhancing neuroplasticity effects.

As discussed above, stimulation source 12 may generate electrical stimulation pulses for the first set 30 and second set 32 of electrodes 18 according to a plurality of stimulation programs, each stimulation program including one or more stimulation sets, each stimulation set specifying suitable stimulation parameters (e.g., amplitude or intensity information, frequency information, phase information, pulse width information, etc.) for electrical stimulation pulses. As discussed below in greater detail, stimulation source 12 may generate and transmit electrical stimulation pulses to first set 30 of electrodes 18 and second set 32 of electrodes 18 substantially simultaneously, in an alternating or multiplexed manner, or in any other suitable manner.

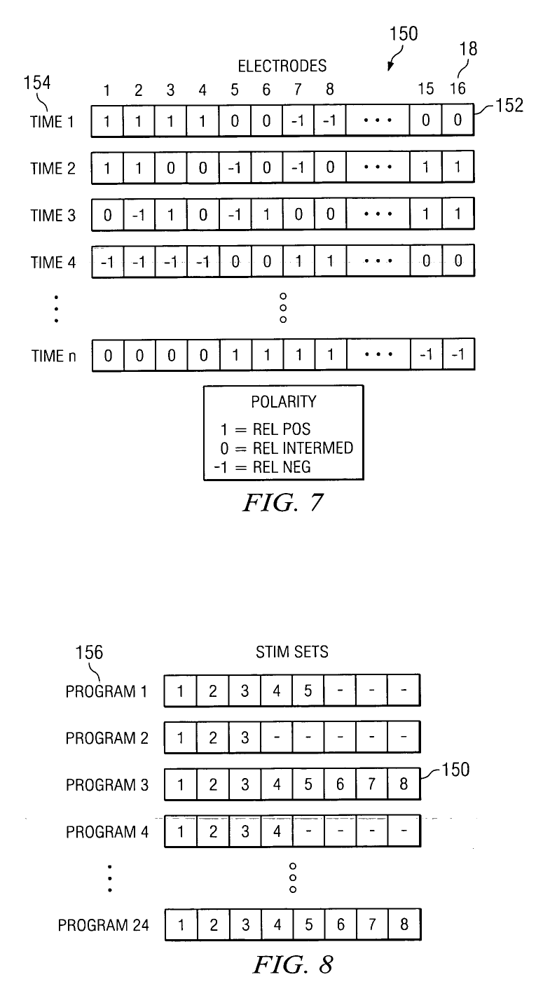

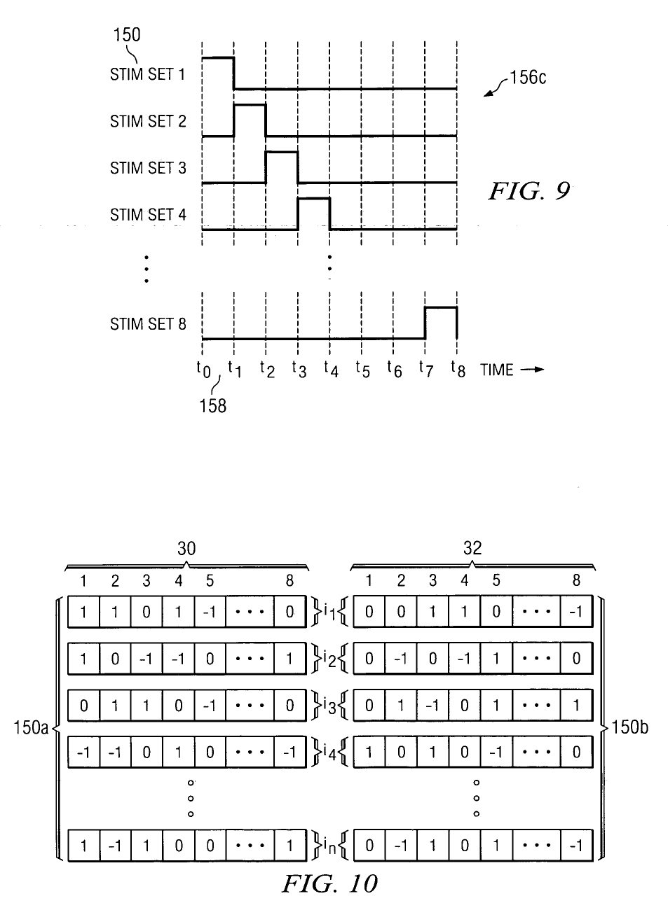

FIG. 7 illustrates an example stimulation set 150 specifying stimulation parameters for electrical stimulation pulses generated by stimulation source 12 and transmitted to one or both of first set 30 of electrodes 18 and second set 32 of electrodes 18. Thus, the following discussion of stimulation sets 150 may apply to electrical stimulation pulses being generated by stimulation source 12 for one or both of first set 30 of electrodes 18 and second set 32 of electrodes 18. One or more stimulation sets 150 may be provided, each stimulation set 150 specifying a number of stimulation parameters for the stimulation set 150. For example, as described more fully below with reference to FIGS. 8-9, multiple stimulation sets 150 may be executed in an appropriate sequence according to a pre-programmed or randomized stimulation program.

Stimulation parameters for a stimulation set 150 may include, for example, amplitude or intensity information, frequency information, phase information, and pulse width information for each of a series of stimulation pulses that electrodes 18 are to deliver to the target nerve tissue 44, 46 during a time interval during which stimulation set 150 is executed, along with a polarity 152 for each electrode 18 in the relevant set 30, 32 of electrodes 18 within each stimulation pulse. In particular embodiments in which a stimulation lead 14 includes two or more stimulation electrodes 18, an electric field may be generated between adjacent or other stimulation electrodes 18 having different polarities 32 to deliver an electrical stimulation pulse to the relevant target nerve tissue 44, 46. One of these stimulation electrodes 18 acts as a cathode and the other of these stimulation electrodes 18 acts as an anode. In particular embodiments in which a stimulation lead 14 includes a single stimulation electrode 18, such as a single stimulation electrode 18 at the tip of stimulation lead 14 for example, an electric field may be generated between the single stimulation electrode 18 and a terminal or other contact associated with stimulation source 12 to deliver an electrical stimulation pulse to the relevant target nerve tissue 44, 46. The single stimulation electrode 18 may act as a cathode and the terminal or other contact associated with stimulation source 12 may act as an anode. Stimulation parameters may also include a pulse shape, for example, biphasic cathode first, biphasic anode first, or any other suitable pulse shape.

For reducing neuroplasticity effects associated with therapeutic electrical stimulation, one or more stimulation parameters for a stimulation set 150 may be randomized or otherwise varied in any suitable manner within the time interval in which stimulation set 150 is executed, spanning one or more stimulation pulses within each stimulation pulse. For example, instead of or in addition to randomizing or otherwise varying polarities 152 for electrodes 18 as described below, the amplitude or intensity information, frequency information, phase information, and pulse width information may be randomized or otherwise varied within predetermined ranges, singly or in any suitable combination, within each stimulation pulse. As another example, instead of or in addition to randomizing or otherwise varying polarities 152 for electrodes 18 over multiple stimulation pulses as described more fully below, the amplitude or intensity information, frequency information, phase information, and pulse width information may be randomized or otherwise varied within predetermined ranges, singly or in any suitable combination, over multiple stimulation pulses, where the combination of stimulation parameters is substantially constant within each stimulation pulse but different for successive stimulation pulses. Such randomization or other variation of stimulation parameters for a stimulation set 150 reduces the ability of the brain to adapt to the neuroplasticity reducing electrical stimulation and dynamically reorganize itself to overcome the effects of the neuroplasticity reducing stimulation.

The polarity for an electrode 18 at a time 154 beginning a corresponding stimulation pulse or sub-interval within a stimulation pulse may be a positive polarity 152 such that current flows out of the electrode 18 into the tissue (from which the current will return through one or more other electrodes 18), a negative polarity 152 such that current flows into the electrode 18 from the tissue (into which the current was delivered from one or more other electrodes 18), or a zero (i.e., “high impedance) polarity such that the electrode 18 is essentially “turned off” and zero or substantially zero current flows out of or into the electrode 18. Thus, the polarity 152 of an electrode determines whether current will flow through the electrode 18 and in which direction. In certain embodiments, the polarity 152 of an electrode 18 may be defined in terms of voltage, in which case the polarity 152 may be relatively positive polarity 152, a relatively negative polarity 152, or an intermediate polarity 152 between the relatively positive polarity 152 and relatively negative polarity 152. For example, the relatively positive polarity 152 may involve a positive voltage, the relatively negative polarity 152 may involve a negative voltage, and the relatively intermediate polarity 152 may involve a zero voltage. As another example, the relatively positive polarity 152 may involve a first negative voltage, the relatively negative polarity 152 may involve a second negative voltage more negative than the first negative voltage, and the relatively intermediate polarity 152 may involve a negative voltage between the first and second negative voltages. The availability of three distinct polarities 152 for an electrode 18 may be referred to as “tri-state” electrode operation. The polarity 152 for each electrode 18 may change for each of the sequence of times 154 corresponding to stimulation pulses or to sub-intervals within a stimulation pulse according to the stimulation parameters specified for the stimulation set 150. For example, as is illustrated in FIG. 7 for an example stimulation set 150 for a stimulation lead 14 with sixteen electrodes 18, the polarities 152 of the sixteen electrodes 18 may change for each of the sequence of times 154. In the example of FIG. 7, a positive or relatively positive polarity 152 is represented using a “1,” a negative or relatively negative polarity 152 is represented using a “−1,” and a zero or relatively intermediate polarity 152 is represented using a “0,” although any suitable values or other representations may be used.

Where appropriate, the polarity 152 for each electrode 18 may change in a predetermined or randomized manner, randomized changes possibly being more effective with respect to any neuroplasticity reducing stimulation for reasons described above.

Where stimulation system 10 provides, in addition to therapeutic electrical stimulation, electrical stimulation to reduce neuroplasticity effects associated with the therapeutic electrical stimulation, each stimulation pulse or sub-interval within a stimulation pulse may be particular to the stimulation being provided; that is, either to therapeutic electrical stimulation or to neuroplasticity reducing electrical stimulation. For example, one or more stimulation pulses or sub-intervals may be designed to provide therapeutic electrical stimulation and one or more other stimulation pulses or sub-intervals may be designed to reduce neuroplasticity effects. In this case, the therapeutic stimulation pulses or sub-intervals and neuroplasticity reducing stimulation pulses or sub-intervals may be arranged temporally in any suitable manner. A therapeutic stimulation pulse or sub-interval may be separated from a successive therapeutic stimulation pulse or sub-interval by any number of neuroplasticity reducing stimulation pulses or sub-intervals and this number may be the same between each pair of therapeutic stimulation pulses or sub-intervals or may vary between each pair of therapeutic stimulation pulses or sub-intervals in a predetermined or randomized manner. As another example, one or more stimulation pulses or sub-intervals may be designed to concurrently provide both therapeutic and neuroplasticity reducing electrical stimulation.

Similarly, where stimulation system 10 provides, in addition to therapeutic electrical stimulation, electrical stimulation to reduce neuroplasticity effects associated with the therapeutic electrical stimulation, each stimulation set 150 may be particular to either the therapeutic electrical stimulation or the neuroplasticity reducing electrical stimulation. For example, one or more stimulation sets 150 may be designed to provide therapeutic electrical stimulation and one or more other stimulation sets 150 may be designed to reduce neuroplasticity effects. In this case, the therapeutic stimulation sets 150 and neuroplasticity reducing stimulation sets 150 may be arranged temporally in any suitable manner. A therapeutic stimulation set 150 may be separated from a successive therapeutic stimulation set 150 by any number of neuroplasticity reducing stimulation sets 150 and this number may be the same between each pair of therapeutic stimulation sets 150 or may vary between each pair of therapeutic stimulation sets 150 in a predetermined or randomized manner. As another example, one or more stimulation sets 150 may be designed to concurrently provide both therapeutic and neuroplasticity reducing electrical stimulation.

In addition, the amplitude or intensity information, frequency information, phase information, or pulse width information for a stimulation set 150 may be particular to the stimulation being provided. For example, therapeutic electrical stimulation may be provided using higher amplitude electrical energy than is used for neuroplasticity reducing electrical stimulation. In this case, the neuroplasticity reducing electrical stimulation may be below the therapeutic target threshold stimulation (i.e. below the threshold where therapeutic electrical stimulation is provided to adjust the level of activity in the target nerve tissue 44, 46 in the person's brain to treat the condition in the person's body corresponding to the target nerve tissue 44, 46). Alternatively, neuroplasticity reducing electrical stimulation may be provided using the same or a higher amplitude electrical energy than is used for therapeutic electrical stimulation (i.e. at or above the threshold where therapeutic electrical stimulation is provided to adjust the level of activity in the target nerve tissue 44, 46 in the person's brain to treat the condition in the person's body corresponding to the target nerve tissue 44, 46). In this case, the neuroplasticity reducing electrical stimulation's primary purpose is not to produce a therapeutic effect, but rather to reduce neuroplasticity. In this manner, the neuroplasticity reducing electrical stimulation could have both a therapeutic and neuroplasticity reducing effect.

FIG. 8 illustrates a number of example stimulation programs 156, each including a number of stimulation sets 150, for electrical stimulation pulses generated by stimulation source 12 and transmitted to one or both of first set 30 of electrodes 18 and second set 32 of electrodes 18. Thus, the following discussion of stimulation sets 150 may apply to electrical stimulation pulses being generated by stimulation source 12 for one or both of first set 30 of electrodes 18 and second set 32 of electrodes 18.

One or more simulation programs 156 may be set up to reduce neuroplasticity effects associated with therapeutic electrical stimulation of the brain. As described above, each stimulation set 150 specifies a number of stimulation parameters for the stimulation set 150. In one embodiment, within each stimulation program 156, stimulation system 10 consecutively executes the sequence of one or more stimulation sets 150 associated with stimulation program 156. The sequence may be executed only once, repeated a specified number of times, or repeated an unspecified number of times within a specified time period. For example, as is illustrated in FIG. 9 for the third example stimulation program 156c including eight stimulation sets 150, each of the eight stimulation sets 150 is consecutively executed in sequence. Although the time intervals 158 (t₁−t₀, t₂−t₁, etc.) during which the stimulation sets 150 are executed are shown as being equal, the present invention contemplates a particular stimulation set 150 being executed over a different time interval 158 than one or more other stimulation sets 150 according to particular needs. One or more stimulation sets 150 within at least one stimulation program 156 may be set up to provide reduced neuroplasticity effects associated with therapeutic electrical stimulation of the brain.

Although stimulation system 10 is illustrated by way of example as accommodating up to twenty-four stimulation programs 156 for a particular set of electrodes 18 (such as the first set 30 or the second set 32 of electrodes 18), each including up to eight stimulation sets 150, the present invention contemplates any appropriate number of stimulation programs 156 each including any appropriate number of stimulation sets 150. For example, in a simple case, a single stimulation program 156 may include a single stimulation set 150, whereas in a more complex case more than twenty-four stimulation programs 156 for a for a particular set of electrodes 18 (such as the first set 30 or the second set 32 of electrodes 18) may each include more than eight stimulation sets 150.

In one embodiment, stimulation system 10 executes only a single stimulation program 156 for the first set 30 of electrodes 18 and a single stimulation program 156 for the second set 32 of electrodes 18 in response to user selection of those stimulation programs 156 for execution. In another embodiment, during a stimulation period, stimulation system 10 executes a sequence of pre-programmed stimulation programs 156 for each of the first set 30 and second set 32 of electrodes 18 until the stimulation period ends. The sequence of pre-programmed stimulation programs 156 for the first set 30 of electrodes 18 and the second set 32 of electrodes 18 may be the same sequence or different sequences of pre-programmed stimulation programs 156. Depending on the length of the stimulation period and the time required to execute a particular sequence of stimulation programs 156, the sequence may be executed one or more times. For example, the stimulation period may be defined in terms of a predetermined number of cycles each involving a single execution of the sequence of stimulation programs 156, the sequence of stimulation programs 156 being executed until the predetermined number of cycles has been completed. As another example, the stimulation period may be defined in terms of time, the sequence of stimulation programs 156 being executed until a predetermined time interval has elapsed or the patient or another user manually ends the stimulation period. Although a sequence of stimulation programs 156 is described, the present invention contemplates a single stimulation program 156 being executed one or more times during a stimulation period according to particular needs. Furthermore, the present invention contemplates each stimulation program 156 being executed substantially immediately after execution of a previous stimulation program 156 or being executed after a suitable time interval has elapsed since completion of the previous stimulation program 156.

Where stimulation system 10 provides, in addition to therapeutic electrical stimulation, electrical stimulation to reduce neuroplasticity effects, each stimulation program 156 may be particular to either the therapeutic electrical stimulation or the neuroplasticity reducing electrical stimulation. For example, one or more stimulation programs 156 may be designed to provide therapeutic electrical stimulation and one or more other stimulation programs 156 may be designed to reduce neuroplasticity effects. In this case, the therapeutic stimulation programs 156 and the neuroplasticity reducing stimulation programs 156 may be arranged temporally in any manner. A therapeutic stimulation program 156 may be separated from a successive therapeutic stimulation program 156 by any number of neuroplasticity reducing stimulation programs 156 and this number may be the same between each pair of therapeutic stimulation programs 156 or may vary between each pair of therapeutic stimulation programs 156 in a predetermined or randomized manner. As another example, one or more stimulation programs 156 may be set up to concurrently provide both therapeutic and neuroplasticity reducing electrical stimulation.

The stimulation programs 156 for reducing neuroplasticity may be used to stimulate the same or different nerve tissue as the stimulation programs 156 used for the therapeutic stimulation. As an example, first set 30 of electrodes 18 of a first location of target nerve tissue in a particular region of the brain is desired, one or more stimulation programs 156 may be set up for the first set 30 of electrodes 18 for therapeutic electrical stimulation of the first target nerve tissue 44 in the particular region and one or more other stimulation programs 156 may be set up for the first set 30 of electrodes 18 for electrical stimulation of the same target nerve tissue 44 in the particular region to reduce neuroplasticity effects associated with the therapeutic electrical stimulation. As another example, one or more stimulation programs 156 may be set up for the first set 30 of electrodes 18 for therapeutic electrical stimulation of first target nerve tissue 44 in a particular region of the brain and one or more other stimulation programs 156 may be set up for the first set 30 of electrodes 18 for electrical stimulation of different nerve tissue in either the same region or in a different region of the brain to reduce neuroplasticity effects associated with the therapeutic electrical stimulation.

As described above, in one embodiment, the nature of any neuroplasticity reducing electrical stimulation may be varied more or less continually, whether in a predetermined or randomized manner, to reduce, prevent, delay, enhance, promote, or otherwise control the ability of the brain to adapt to the neuroplasticity reducing electrical stimulation and dynamically reorganize itself accordingly. In a more particular embodiment, where the neuroplasticity reducing electrical stimulation is provided concurrently with therapeutic electrical stimulation, the neuroplasticity reducing electrical stimulation may be randomized or otherwise varied about the therapeutic electrical stimulation to achieve this result. In essence, the randomized or otherwise varied neuroplasticity reducing electrical stimulation makes it more difficult for the brain to dynamically reorganize itself to overcome the effects of the therapeutic electrical stimulation.

Stimulation programs 156 (or stimulation sets 150) for the first set 30 of electrodes 18 may be executed substantially simultaneously with stimulation programs 156 (or simulation sets 150) for the second set 32 of electrodes 18, may be alternated, or multiplexed, with stimulation programs 156 (or stimulation sets 150) for the second set 32 of electrodes 18, or may be arranged in any other suitable manner with respect to stimulation programs 156 (or stimulation sets 150) for the second set 32 of electrodes 18.

FIG. 10 illustrates an example of a stimulation set 150a of a stimulation program 156 for a first set 30 of eight electrodes 18 being executed substantially simultaneously with a stimulation set 150b of a stimulation program 156 for a second set 32 of eight electrodes 18 according to one embodiment of the invention. As discussed above with reference to FIG. 7, stimulation sets 150a and 150b specify stimulation parameters for electrical stimulation pulses generated by stimulation source 12 and transmitted to first set 30 of electrodes 18 and second set 32 of electrodes 18, respectively. At each time interval i₁-iₙ, stimulation pulses are substantially simultaneously generated by stimulation source 12 for both the first set 30 of electrodes 18 and the second set 32 of electrodes 18 according to the stimulation parameters specified by stimulation sets 150a and 150b, respectively. Stimulation sets 150a and 150b may specify any number of similar or different stimulation parameters.

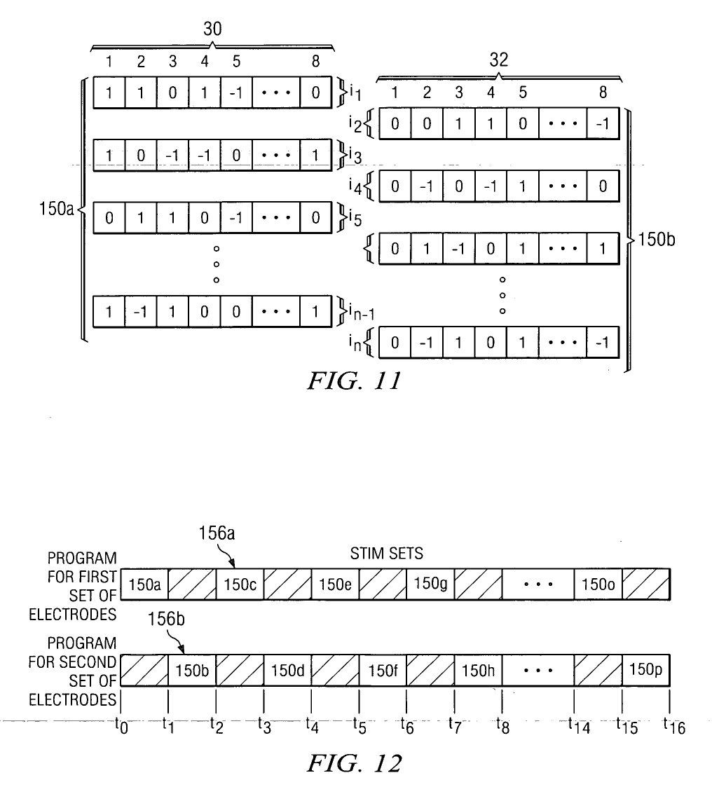

FIG. 11 illustrates an example of a stimulation set 150a of a stimulation program 156 for a first set 30 of eight electrodes 18 being executed in an alternating, or multiplexed, manner with a stimulation set 150b of a stimulation program 156 for a second set 32 of eight electrodes 18 according to one embodiment of the invention. As discussed above, stimulation sets 150a and 150b specify stimulation parameters for electrical stimulation pulses generated by stimulation source 12 and transmitted to first set 30 of electrodes 18 and second set 32 of electrodes 18, respectively. At each time interval i₁-iₙ, stimulation pulses are generated by stimulation source 12 for either the first set 30 of electrodes 18 or the second set 32 of electrodes 18 according to the stimulation parameters specified by the respective stimulation set 150a or 150b. Thus, stimulation pulses may be generated for the first set 30 and the second set 32 of electrodes 18 in an alternating, or multiplexed, manner.

FIG. 12 illustrates an example of multiple stimulation sets 150 of a first stimulation program 156a for a first set 30 of electrodes 18 being executed in an alternating, or multiplexed, manner with multiple stimulation sets 150 of a second stimulation program 156b for a second set 32 of electrodes 18 according to one embodiment of the invention. At each time interval (t₀-t₁, t₁-t₂ etc.), either a stimulation set 150 of first stimulation program 156a for the first set 30 of electrodes 18 or a stimulation set 150 of second stimulation program 156b for the second set 32 of electrodes 18 is executed. For example, a first stimulation set 150a of first stimulation program 156a is executed in the time interval t₀-t₁, followed by a first stimulation set 150b of second stimulation program 156b executed in the time interval t₁-t₂, followed by a second stimulation set 150c of first stimulation program 156a executed in the time interval t₂-t₃, and so on. In this manner, complete stimulation sets 150 may be executed in an alternating, or multiplexed, manner.

Although FIGS. 10-12 illustrate various examples of the execution of stimulation programs 156 (or stimulation sets 150) for the first set 30 and the second set 32 of electrodes 18 relative to each other, the execution of stimulation programs 156 (or stimulation sets 150) for the first set 30 of electrodes 18 may be arranged in any other suitable manner with respect to stimulation programs 156 (or stimulation sets 150) for the second set 32 of electrodes 18.

The present invention contemplates any suitable circuitry within stimulation source 12 for generating and transmitting electrical stimulation pulses for electrically stimulating target nerve tissue at multiple locations in a person's brain to treat multiple conditions in the person's body and, where appropriate, to reduce, enhance, or otherwise modify neuroplasticity effects in the person's brain, whether separate from or concurrently with the therapeutic electrical stimulation. Example circuitry that may be used is illustrated and described in U.S. Pat. No. 6,609,031 B1, the subject matter of which is hereby incorporated by reference herein as if fully illustrated and described herein.

Although the present invention has been described above in connection with several embodiments, a number of changes, substitutions, variations, alterations, transformations, and modifications may be suggested to one skilled in the art, and it is intended that the present invention encompass such changes, substitutions, variations, alterations, transformations, and modifications as fall within the spirit and scope of the appended claims.

What is claimed is:

1. An electrical stimulation system for electrically stimulating target nerve tissue at multiple locations in the brain to treat multiple conditions in the body, comprising:
one or more first electrodes adapted to be implanted inside a person's skull and positioned proximate first target nerve tissue in the brain associated with a first condition in the person's body and to deliver first electrical stimulation pulses to the first target nerve tissue according to one or more first stimulation programs to treat the first condition in the person's body; and
one or more second electrodes adapted to be implanted inside a person's skull and positioned proximate second target nerve tissue in the brain associated with a second condition in the person's body and to deliver second electrical stimulation pulses to the second target nerve tissue according to one or more second stimulation programs to treat the second condition in the person's body; and
a stimulation source operable to:
generate the first electrical stimulation pulses according to the one or more first stimulation programs for transmission to the one or more first electrodes to cause the one or more first electrodes to deliver the first electrical stimulation pulses to the first target nerve tissue to treat the first condition in the person's body; and
generate the second electrical stimulation pulses according to the one or more second stimulation programs for transmission to the one or more second electrodes to cause the one or more second electrodes to deliver the second electrical stimulation pulses to the second target nerve tissue to treat the second condition in the person's body.

2. The system of claim 1, wherein:
the first target nerve tissue in the brain comprises nerve tissue at a first location in the brain; and
the second target nerve tissue in the brain comprises nerve tissue at a second location in the brain different from the first location in the brain.

3. The system of claim 2, wherein the first target nerve tissue in the brain comprises nerve tissue associated with a first one of the following and the second target nerve tissue in the brain comprises nerve tissue associated with a second one of the following:
the frontal lobe;
the occipital lobe;
the parietal lobe;
the temporal lobe;
the cerebellum; and
the brain stem.

4. The system of claim 2, wherein the first target nerve tissue in the brain comprises nerve tissue associated with a first one of the following and the second target nerve tissue in the brain comprises nerve tissue associated with a second one of the following:
the primary auditory cortex;
the secondary auditory cortex;
the remote projection region;
Brodman's area 41;
Brodman's area 42;
Brodman's area 22; and
Brodman's area 52.

5. A method for electrically stimulating target nerve tissue in the brain to treat a condition in the body, comprising:
using a stimulation source to generate first electrical stimulation pulses according to one or more first stimulation programs for transmission to a plurality of first electrodes implanted inside the person's skull to cause the first electrodes to deliver the first electrical stimulation pulses to the first target nerve tissue in the person's brain associated with a first condition in the person's body;
using the stimulation source to generate second electrical stimulation pulses according to one or more second stimulation programs for transmission to a plurality of second electrodes implanted inside the person's skull to cause the second electrodes to deliver the second electrical stimulation pulses to the second target nerve tissue in the person's brain associated with a second condition in the person's body;
in response to receiving the first electrical stimulation pulses transmitted from the stimulation source, using the first implanted electrodes to deliver the first electrical stimulation pulses to the first target nerve tissue in the brain according to the one or more first stimulation programs to treat the first condition in the person's body; and
in response to receiving the second electrical stimulation pulses transmitted from the stimulation source, using the second implanted electrodes to deliver the second electrical stimulation pulses to the second target nerve tissue in the brain according to the one or more second stimulation programs to treat the second condition in the person's body.

6. The method of claim 5, wherein:
the first target nerve tissue in the brain correlates to a first painful region of the person's body; and
the first electrical stimulation pulses are delivered to the first target nerve tissue in the brain to adjust a level of activity in the first target nerve tissue in the brain to provide pain relief to the first painful region of the person's body.

7. The method of claim 6, wherein:
the second target nerve tissue in the brain correlates to a second painful region of the person's body; and
the second electrical stimulation pulses are delivered to the second target nerve tissue in the brain to adjust a level of activity in the second target nerve tissue in the brain to provide pain relief to the second painful region of the person's body.

8. The method of claim 5, wherein:
the first target nerve tissue is overactive; and
the first electrical stimulation pulses are delivered to the first target nerve tissue in the brain to decrease activity in the first target nerve tissue to treat the first condition in the person's body.

9. The method of claim 5, wherein:
the first target nerve tissue is underactive; and
the first electrical stimulation pulses are delivered to the first target nerve tissue in the brain to increase activity in the first target nerve tissue to treat the first condition in the person's body.

10. The method of claim 5, wherein the one or more first electrodes are implanted into an extradural region adjacent the brain.

11. The method of claim 5, wherein the first and second conditions in the person's body are substantially unrelated.

12. The method of claim 5, wherein:
the first target nerve tissue in the brain comprises nerve tissue at a first location in the brain; and
the second target nerve tissue in the brain comprises nerve tissue at a second location in the brain different from the first location in the brain.

13. The method of claim 12, wherein the first target nerve tissue in the brain comprises nerve tissue associated with a first one of the following and the second target nerve tissue in the brain comprises nerve tissue associated with a second one of the following:
the frontal lobe;
the occipital lobe;
the parietal lobe;
the temporal lobe;
the cerebellum; and
the brain stem.

14. The method of claim 12, wherein the first target nerve tissue in the brain comprises nerve tissue associated with a first one of the following and the second target nerve tissue in the brain comprises nerve tissue associated with a second one of the following:
the primary auditory cortex;
the secondary auditory cortex;
the remote projection region;
Brodman's area 41;
Brodman's area 42;
Brodman's area 22; and
Brodman's area 52.

15. The method of claim 5, wherein:
each of the one or more first stimulation programs comprises one or more first stimulation sets, each first stimulation set specifying a plurality of first stimulation parameters; and
each of the one or more second stimulation programs comprises one or more second stimulation sets, each second stimulation set specifying a plurality of second stimulation parameters.

16. The method of claim 15, wherein:
the first stimulation parameters for a particular first stimulation set comprise a polarity for each of the one or more first electrodes at each of one or more times within a stimulation pulse for that particular first stimulation set; and
the second stimulation parameters for a particular second stimulation set comprise a polarity for each of the one or more second electrodes at each of one or more times within a stimulation pulse for that particular second stimulation set.

17. The method of claim 15, wherein:
at least one first stimulation set accomplishes electrical stimulation of the first target nerve tissue in the brain for treating the first condition in the person's body; and
at least one other first stimulation set accomplishes electrical stimulation of the first target nerve tissue in the brain for modifying neuroplasticity.

18. The method of claim 17, wherein modifying neuroplasticity effects in the person's brain comprises reducing neuroplasticity effects in the person's brain.

19. The method of claim 17, wherein modifying neuroplasticity effects in the person's brain comprises enhancing neuroplasticity effects in the person's brain.

20. The method of claim 15, comprising using the stimulation source to generate, substantially concurrently with the generation of the first electrical stimulation pulses for treating the first condition in the person's body, electrical stimulation pulses for transmission to the one or more first electrodes of the lead to cause the one or more first electrodes to deliver electrical stimulation pulses to the first target nerve tissue in the brain to treat neuroplasticity effects associated with the condition-treating electrical stimulation of the first target nerve tissue in the brain, the neuroplasticity-treating electrical stimulation being randomized about the condition-treating electrical stimulation, the neuroplasticity-treating electrical stimulation making it more difficult for the brain to dynamically reorganize itself to overcome effects of the condition-treating electrical stimulation;
wherein one or more first stimulation sets accomplish electrical stimulation of the first target nerve tissue in the brain for treating the first condition in the person's body and one or more other first stimulation sets accomplish electrical stimulation of the first target nerve tissue in the brain for treating neuroplasticity, each condition-treating electrical stimulation set being separated from a next condition-treating electrical stimulation set by a number of neuroplasticity-treating stimulation sets that is greater than or equal to zero.
